(19) United States
(12) Patent Application Publication
(10) Pub. No.: US 2026/0094714 A1
(43) Pub. Date: Apr. 2, 2026

(54) SYSTEMS AND METHODS FOR DIAGNOSING HEPATOCELLULAR CARCINOMA BASED ON THE DETECTION AND INTERPRETATION OF A PANEL OF MICRORNAS IN A SUBJECT

(71) Applicant: City University of Hong Kong, Hong Kong (HK)

(72) Inventors: Mengsu YANG, Hong Kong (HK); Kelvin Ka Ki NG, Hong Kong (HK)

(21) Appl. No.: 19/337,843

(22) Filed: Sep. 23, 2025

Related U.S. Application Data
(60) Provisional application No. 63/701,559, filed on Sep. 30, 2024.

(51) Int. Cl. G16H 50/20; C12Q 1/6806; C12Q 1/686; C12Q 1/6883; G01N 33/68; G06N 20/00; G16B 25/10; G16B 40/20; G16H 15/00; G16H 50/30; G16H 50/70

(52) U.S. Cl. CPC G16H 50/20; C12Q 1/6806; C12Q 1/686; C12Q 1/6883; G01N 33/6893; G06N 20/00; G16B 25/10; G16B 40/20; G16H 15/00; G16H 50/30; G16H 50/70; C12Q 2600/178; G01N 2333/471

(57) ABSTRACT

A system for generating a hepatocellular carcinoma (HCC) report for a subject based on blood-based molecular profiling is provided. The system includes a sample preparation module configured to process a blood sample by isolating both plasma and serum fractions. A nucleic acid extraction module extracts a microRNA (miRNA) profile from the plasma fraction, and a real-time PCR module detects and quantifies the expression levels of a predefined panel of miRNAs, including miR-361-5p, miR-130a-3p, miR-27a-3p, miR-30d-5p, and miR-193a-5p. The expression levels of the predefined panel of miRNAs are used by a trained machine learning classifier to generate a HCC risk classification categorizing the subject into one of HCC risk levels. An alpha-fetoprotein (AFP) level detected and quantified from the serum fraction may also be used in combination with the miRNA expression levels in the HCC risk classification for enhanced prediction accuracy.

### CROSS-REFERENCE TO RELATED APPLICATIONS

The present application claims priority from U.S. Provisional Utility Patent application No. 63/701,559 filed Sep. 30, 2024; the disclosure of which is incorporated herein by reference in its entirety.

### FIELD OF THE INVENTION

The present invention generally relates to the fields of molecular biology and computational pathology. More specifically, the present invention relates to systems and methods for generating a hepatocellular carcinoma report of a subject using circulating microRNA biomarkers and machine learning-based classification.

### BACKGROUND OF THE INVENTION

Hepatocellular carcinoma (HCC) is the most common type of primary liver cancer, accounting for more than 90% of all liver cancer cases. A substantial proportion—approximately 80% to 90%—of newly diagnosed HCC patients have preexisting liver cirrhosis (LC). Among the various etiological factors, chronic hepatitis B virus (HBV) infection is the predominant risk factor, particularly in Asia, where it is responsible for over 50% of HCC cases. Globally, HCC ranks as the third leading cause of cancer-related mortality. Despite this, early-stage HCC is potentially curable, with 5-year survival rates exceeding 70% following interventions such as liver ablation, surgical resection, or transplantation. However, the prognosis for HCC remains poor in many cases due to the absence of reliable and effective screening methods for early detection. As a result, approximately 50% of patients are diagnosed at advanced stages, where curative options are no longer viable. Unlike many other solid tumors, the diagnosis of HCC typically relies on non-invasive imaging modalities rather than tissue biopsy, due to concerns regarding hemorrhage and tumor seeding. Current clinical guidelines from the American Association for the Study of Liver Diseases (AASLD) recommend biannual surveillance of at-risk populations, particularly adults with liver cirrhosis, using liver ultrasound in combination with serum alpha-fetoprotein (AFP) testing. AFP is the most widely adopted serological biomarker for HCC screening worldwide. An abnormal surveillance result—such as a liver nodule measuring ≥10 mm or an elevated AFP level above 20 ng/ml (AFP20)—triggers further diagnostic evaluation through multiphase computed tomography (CT) or magnetic resonance imaging (MRI).

In recent years, liquid biopsy has emerged as a promising, minimally invasive approach for early cancer detection and longitudinal disease monitoring. MicroRNAs (miRNAs), a class of small non-coding RNAs approximately 22 nucleotides in length, regulate gene expression at the post-transcriptional level by binding to 3′ untranslated regions (3′ UTRs) of target messenger RNAs (mRNAs), resulting in mRNA degradation or translational repression. The exceptional stability of circulating miRNAs in blood makes them attractive candidates for use as biomarkers in liquid biopsy applications. Numerous efforts have been made to identify miRNA signatures for HCC diagnosis; however, findings across different studies have been inconsistent. For example, circulating miR-122-5p has shown variable patterns of dysregulation in HCC, with some studies reporting upregulation, while others have observed downregulation or no significant change.

Accordingly, the present invention provides the methods and systems for analyzing a panel of novel miRNAs associated with HCC in a subject sample, wherein the method is implemented through a defined set of molecular processing and data analysis steps to enable improved clinical decision-making. The invention further relates to an integrated workflow or platform that facilitates the detection and interpretation of microRNA expression profiles, thereby enhancing the utility of circulating biomarkers in managing liver disease progression.

### SUMMARY OF THE INVENTION

It is an objective of the present invention to provide the system and method to solve the aforementioned technical problems.

In accordance with a first aspect of the present invention, a system for diagnosing HCC in a subject is provided. Specifically, the system includes: a sample preparation module configured to receive a blood sample and isolate a plasma fraction and a serum fraction therefrom; a nucleic acid extraction module configured to extract a miRNA profile from the plasma fraction; a real-time PCR module configured to detect and quantify expression levels of a panel of miRNAs comprising miR-361-5p, miR-130a-3p, miR-27a-3p, miR-30d-5p, and miR-193a-5p in the miRNA profile; a computing device comprising a processor and memory storing instructions that, when executed, cause the processor to: receive as input the quantified expression levels of the panel of miRNAs; process the input using a trained classifier to generate, from the expression levels of the panel of miRNAs, a HCC risk classification categorizing the subject into one of the HCC risk levels (i.e., low risk, indeterminate risk, and high risk).

In accordance with another embodiment, the computing device is further configured to receive an AFP level detected and quantified from the serum fraction and compare it to a threshold of 20 ng/ml. Regardless of whether the AFP levels are below or above the threshold, the classifier incorporates AFP as a binary input together with the expression levels of the panel of miRNAs to classify the HCC risk. Alternatively, when AFP data are unavailable, a separate classifier using the same five miRNA targets alone is applied, which classifies subjects with slightly reduced performance.

In accordance with yet another embodiment, the trained classifier includes a logistic regression model trained using annotated training datasets comprising miRNA expression profiles and ground-truth HCC diagnoses. The training datasets may be obtained from patient medical records.

In accordance with yet another embodiment, the system further includes a training module configured to update the trained classifier based on new annotated training datasets.

In accordance with yet another embodiment, the subject is a liver cirrhosis patient.

In accordance with a second aspect of the present invention, a computer-implemented method for diagnosing HCC in a subject is provided. Particularly, the method includes: obtaining a blood sample from a subject; isolating a plasma fraction from the blood sample; extracting a miRNA profile of the subject from the plasma fraction; detecting and quantifying expression levels of a panel of miRNAs comprising miR-361-5p, miR-130a-3p, miR-27a-3p, miR-30d-5p, and miR-193a-5p in the miRNA profile; processing the quantified expression levels with a trained machine learning classifier implemented on a computer processor, wherein the classifier is configured to generate, from the expression levels of the panel of miRNAs, a HCC risk classification categorizing the subject into one of HCC risk levels (i.e., low risk, indeterminate risk, and high risk).

In accordance with one embodiment, the method further includes detecting and quantifying an AFP level from a serum fraction isolated from the same blood sample; comparing the AFP level to a threshold of 20 ng/mL. Regardless of whether the AFP levels are below or above the threshold, the classifier incorporates AFP as a binary input together with the expression levels of the panel of miRNAs to classify the HCC risk. Alternatively, when AFP data are unavailable, a separate classifier using the same five miRNA targets alone is applied, which classifies subjects with slightly reduced performance.

In accordance with yet another embodiment, the subject is a liver cirrhosis patient.

In accordance with yet another embodiment, the classifier includes a logistic regression model trained using LASSO regularization on annotated training datasets.

In accordance with yet another embodiment, the method further comprises generating, by the computer processor, a HCC report including information on the HCC risk classification. The HCC report may further include one or more recommendations for: repeat testing, multiphase contrast-enhanced MRI, or oncology consultation.

### BRIEF DESCRIPTION OF THE DRAWINGS

Embodiments of the invention are described in more details hereinafter with reference to the drawings, in which:

FIGS. 1A-IC depict the upregulation of 18 miRNAs in HCC patients, in which FIG. 1A illustrates the workflow of the present invention; FIG. 1B shows the volcano plot of differentially expressed miRNAs between HCC patients and LC patients in the discovery stage, and 16 validated miRNA targets are annotated; FIG. 1C demonstrates the unsupervised hierarchical clustering of 18 miRNA targets across 354 matched samples in validation stage 1;

FIGS. 2A-2D depict the detection performance and risk score of combined panel across different sample sets, in which FIG. 2A relates to the training set; FIG. 2B illustrates the testing set; FIG. 2C displays the validation set; and FIG. 2D shows the risk score distribution of combined panel across patients with different HCC stages, revealing that the combined panel demonstrates superior performance compared to AFP20 alone across all sample sets;

FIGS. 3A-3E depict the association of miRNAs with clinical parameters, in which FIG. 3A-FIG. 3C respectfully show the association with different HCC stages (FIG. 3A), tumor size (FIG. 3B), and tumor invasion (FIG. 3C); FIG. 3D illustrates the interaction network of the 5 miRNAs in the panel and their target genes; and FIG. 3E displays the Kyoto Gene and Genome Encyclopedia (KEGG) enrichment analysis for target genes of the 5-miRNA panel;

FIG. 4 depicts a system of diagnosing HCC in a subject in according to one embodiment of the present invention.

### DETAILED DESCRIPTION

In the following description, systems and/or methods of diagnosing HCC in a subject and the likes are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

As used herein, the term “annotated training dataset” refers to a collection of biological and/or clinical data obtained from human patient subjects; wherein each data entry is labeled with relevant clinical annotations. These datasets typically include molecular or biomarker profiles. The annotations serve as ground truth for supervised machine learning model training and validation.

In some embodiments, annotated training datasets may be derived from prospective or retrospective clinical studies, multi-center clinical trials, hospital biorepositories, or publicly available databases such as the Gene Expression Omnibus (GEO), The Cancer Genome Atlas (TCGA), or the International Cancer Genome Consortium (ICGC). The datasets may be preprocessed to remove noise or artifacts and may be normalized to ensure consistent comparison across samples. In certain embodiments, the annotated dataset comprises at least miRNA expression values of a panel of interest (e.g., miR-361-5p, miR-130a-3p, miR-27a-3p, miR-30d-5p, and miR-193a-5p) along with ground-truth diagnostic outcomes (e.g., confirmed HCC vs. non-HCC diagnosis).

These annotated datasets are useful for training, testing, and validating classifiers, including logistic regression models, neural networks, random forests, and other supervised learning algorithms, to assess the risk or likelihood of HCC in patients. The term may also encompass curated datasets that include interplate controls, normalization factors, and metadata linking each sample to a clinical cohort or outcome category.

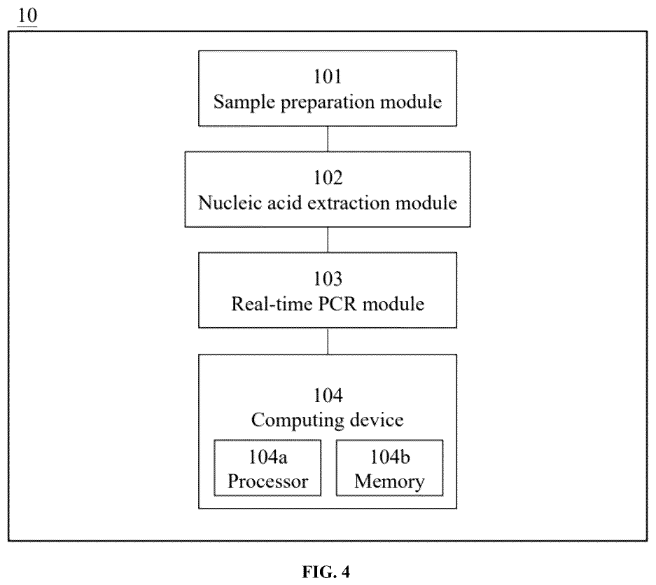

In accordance with a first aspect of the present invention, a system for diagnosing HCC in a subject through the integration of molecular profiling and computational processing is provided. Referring to FIG. 4, the system 10 is designed to streamline the early detection and classification of HCC risk by analyzing specific circulating miRNAs in blood samples using high-throughput, real-time quantitative PCR and machine learning-based decision support.

In one embodiment, the system 10 includes a sample preparation module 101 configured to receive a blood sample obtained from a subject, preferably via venipuncture. Upon receipt, the module 101 processes the blood sample to separate it into its plasma and serum fractions through centrifugation or another standard separation technique. These distinct fractions are used downstream for molecular profiling and biomarker analysis.

From the plasma fraction, a nucleic acid extraction module 102 is employed to isolate total RNA, including miRNA species. This module 102 may utilize spin column-based methods, magnetic bead technology, or cartridge-based automation to ensure the efficient and high-quality extraction of nucleic acids suitable for downstream analysis. The focus of extraction is on the plasma miRNA profile, which is known to contain stable, circulating miRNA biomarkers relevant to tumor biology.

The extracted RNA is then directed to a real-time PCR module 103, which is configured to detect and quantify the expression levels of a pre-defined panel of miRNAs comprising miR-361-5p, miR-130a-3p, miR-27a-3p, miR-30d-5p, and miR-193a-5p. The panel has been identified as particularly informative for distinguishing HCC from non-HCC liver pathologies, especially among at-risk populations. The real-time PCR module 103 includes thermal cycling capability and fluorescence detection optics that support locked nucleic acid (LNA) probe-based quantification, enabling sensitive and specific amplification of the targeted miRNAs.

The system 10 further includes a computing device 104 with a processor 104a and a memory 104b storing executable instructions. When the instructions are executed, the processor is configured to receive as input the quantified expression levels of the five target miRNAs from the PCR module. The processor then processes this input using a trained classifier—such as a logistic regression model—to generate a HCC risk classification categorizing the subject into one of HCC risk levels (i.e., low risk, indeterminate risk, and high risk).

The classifier is trained using annotated training datasets, which include miRNA expression profiles along with corresponding clinical HCC diagnoses, allowing for predictive modeling and risk stratification.

The computing device may also output a HCC report, which is displayed via a user interface. The HCC report may include information on the HCC risk classification and the relative expression of each miRNA in the panel. This HCC report assists clinicians in making informed decisions regarding further diagnostic evaluation, treatment initiation, or surveillance strategies.

In some embodiments, the computing device is further configured to integrate the miRNA expression data with the AFP level measured from the serum fraction. The AFP level, a widely used biomarker for liver cancer screening, is processed alongside the miRNA panel to enhance accuracy. The system includes logic for comparing the AFP concentration to a clinically established threshold—typically 20 ng/ml—as part of the classification decision-making. The inclusion of both molecular and serological markers allows for multimodal risk assessment.

To ensure adaptability and continual improvement of diagnostic performance, the system may further include a training module configured to retrain or update the classifier as new annotated training datasets become available. This dynamic learning capability ensures that the model remains current with emerging clinical data and variations in population-specific biomarker profiles.

The system is particularly suitable for deployment in populations at elevated risk for HCC, such as individuals diagnosed with LC. Since LC patients are typically monitored over time for progression to HCC, the described system provides a minimally invasive, high-precision tool for early-stage detection and surveillance.

Through the combination of blood-based molecular diagnostics, machine learning-based classification, and real-time clinical decision support, the system of the present invention enables improved identification of HCC in at-risk individuals, supporting timely and personalized clinical intervention.

In accordance with a second aspect of the present invention, a computer-implemented method for diagnosing HCC in a subject through analysis of circulating miRNA profiles and optional integration of serological biomarkers is provided. The method leverages machine learning-based predictive modeling to support early detection and risk stratification of HCC in clinical settings.

The process begins by obtaining a blood sample from a subject, preferably through routine venipuncture. From the collected blood, the method includes isolating a plasma fraction using standard centrifugation or plasma separation protocols. The isolated plasma is then subjected to nucleic acid extraction, focusing on recovering small RNA species, particularly miRNAs, which serve as minimally invasive biomarkers of tumorigenesis.

A miRNA profile of the subject is extracted from the plasma fraction, and the expression levels of a specific panel of miRNAs are quantified. This panel includes miR-361-5p, miR-130a-3p, miR-27a-3p, miR-30d-5p, and miR-193a-5p, which have been identified as informative indicators in differentiating HCC from non-HCC liver conditions such as liver cirrhosis. The quantification step may be carried out using RT-qPCR with locked nucleic acid (LNA) probes or any suitable quantitative platform capable of providing expression values suitable for computational input.

Once quantified, the expression values of the five miRNAs are processed by a trained machine learning classifier, which is implemented on a computer processor. This classifier, in one embodiment, comprises a logistic regression model trained using least absolute shrinkage and selection operator (LASSO) regularization, which allows the model to select the most informative features while avoiding overfitting. The classifier is trained on annotated training datasets, which include ground-truth diagnostic labels and matched molecular profiles, enabling the model to learn discriminative patterns associated with HCC risk.

Upon processing the input data, the classifier generates a HCC risk classification categorizing the subject into one of HCC risk levels (i.e., low risk, indeterminate risk, and high risk). Based on this expression report, the computer processor may generate an HCC report, which may include one or more of the following outputs: the HCC risk classification, an expression report, which indicates the relative expression levels of the miRNA panel, a diagnostic interpretation, or suggested next steps. The suggested next steps may include clinical actions such as conducting repeated testing, ordering a multiphase contrast-enhanced MRI, or referring the patient for oncology consultation, thereby supporting timely clinical intervention and care planning.

In another embodiment, the relative expression level of each miRNA is determined by normalizing the raw quantification values—typically cycle threshold (Ct) values from real-time PCR—against an internal control, such as miR-16-5p, which exhibits stable expression across different samples and disease states. The resulting normalized values (e.g., using the 2^{-ΔCt} method, where ΔCt=Ct_{target}−Ct_{treference}) reflect the abundance of each target miRNA in the subject's plasma relative to the reference miRNA within the same sample.

The HCC report is provided through a user interface, which may be integrated into a clinical dashboard, electronic medical record system, or a stand-alone web portal. The interface is designed to be accessible and interpretable by healthcare providers.

In some embodiments, the method further includes the detection and quantification of AFP levels from the serum fraction of the same blood sample. The quantified AFP value may be combined with the expression levels of the panel of miRNAs in the HCC risk classification to enhance its predictive accuracy. In these embodiments, the AFP measurement may be compared to a threshold value of 20 ng/mL, which is commonly used in clinical practice as a cutoff for elevated HCC risk.

The subject undergoing testing is preferably a LC patient, as individuals with LC are at heightened risk for developing HCC and are often the target population for surveillance programs.

Overall, the method provides a scalable and interpretable framework for the early identification of HCC using circulating biomarkers and machine learning models. It offers the ability to personalize diagnostic recommendations and supports clinical workflows by enabling integration with traditional serologic markers and imaging-based follow-up protocols.

### EXAMPLES

### Example 1: Identifying Upregulated miRNAs in HCC Patients

A total of 522 non-hemolyzed plasma samples are collected from the Chongqing (CQ) and Beijing (BJ) cohorts. Briefly, in this multicenter cohort study, plasma samples are collected from a total of 522 patients (male:female=344:178; median age: 56 years old, range: 17-86) across three stages: discovery stage, validation stage 1 and validation stage 2. In the discovery stage, microarray analysis is performed on 90 samples (50 patients with HCC and 40 patients with LC) to identify differentially expressed miRNAs between HCC patients and LC patients. In the validation stage 1, significant miRNAs from the discovery stage are selected and validated with reverse transcription RT-qPCR in a total of 354 matched samples, including 72 samples from the discovery stage and an additional 282 samples. Blood samples for the discovery stage and validation stage 1 are collected from a CQ cohort from the Second Affiliated Hospital of Chongqing Medical University (N=227) and a BJ cohort from Chinese PLA General Hospital (N=151) from November 2020 to April 2022. An additional 150 samples are recruited from the Second Affiliated Hospital of Chongqing Medical University from September 2022 to December 2022 as validation stage 2. Most patients had a history of chronic HBV. HCC patients are diagnosed through computed tomography (CT) or magnetic resonance imaging (MRI), part of which are further confirmed histologically by at least two independent histopathologists according to the American Association of the Study of Liver Diseases (AASLD) guidelines. Tumor staging is classified according to the Barcelona Clinic Liver Cancer (BCLC) staging system, with stage 0 and stage categorized as early-stage HCC and the remaining stages categorized as late-stage HCC. None of the patients with HCC has previously undergone surgical operation, chemotherapy or radiotherapy before blood collection. Patients with LC are diagnosed with ultrasound and included as at-risk controls. Chronic HBV infection is defined as chronic liver disease caused by persistent HBV infection (positive HBsAg>6 months with detectable serum HBV DNA). Clinical characteristics are collected for subsequent analysis. Informed consent is obtained from all patients. The experiment is approved by the Hospital Ethical Committee of the Second Affiliated Hospital of Chongqing Medical University (2022-80) and adhered to the principles of the Helsinki Declaration.

Peripheral blood samples are collected from all subjects using 4-mL BD Vacutainer EDTA tubes (Becton Dickinson) and stored at 4° C. Plasmas are isolated within 8 hours from collection and centrifuged at 4,000 rpm for 15 mins at 4° C. Plasma samples are aliquoted in sterilized Eppendorf tubes and stored at −80° C. until RNA extraction. The absorbance at 414 nm (A414) is measured for all plasma samples using NanoDrop ND-2000 (Thermo Scientific) to evaluate the degree of hemolysis. Plasma samples with A414 value greater than 0.2 are removed from downstream analysis.

For microarray analysis, miRNA is extracted from 400 μl plasma using mirVana miRNA Isolation Kit (Thermo Scientific) following the manufacturer's instructions. Total RNA is quantified by NanoDrop ND-2000 (Thermo Scientific) and RNA integrity is assessed by Agilent Bioanalyzer 2100 (Agilent Technologies). For qPCR analysis, miRNA is extracted from 200 μl plasma using miRNeasy Serum/Plasma Kit (Qiagen). The extraction is performed according to the manufacturer instructions with minor modifications: 1) to normalize the extraction variability, a spike in control is added to the sample during the RNA isolation phase; 2) to improve RNA isolation efficiency, bacteriophage MS2 carrier RNA (Thermo Scientific) is added to the sample during the RNA isolation phase (1 μg per ml of QIAzol). Extracted RNAs are stored at −80° C. until further use.

The clinical and demographic data of the study participants are summarized in Table 1. 354 matched patients, adjusted for confounding factors including age, sex, and HBV status, are included in the validation stage 1. Sample matching is not employed in the validation stage 2 to more accurately represent the actual clinical setting, resulting in a higher proportion of males, older individuals and patients with HBV.

TABLE 1

| | | | | Validation stage 2 | |
|---|---|---|---|---|---|
| | | | | Additional | |
| Matched samples in validation stage 1 | | | | validation set | |
| CQ cohort (n = 203) | | BJ cohort (n = 151) | | (n = 150) | |
| HCC | LC | HCC | LC | HCC | LC |
| (n = 94) | (n = 109) | (n = 83) | (n = 68) | (n = 29) | (n = 121) |

Sex

| Male | 71 | 80 | 58 | 54 | 26 | 43 |
|---|---|---|---|---|---|---|
| | (76%) | (73%) | (70%) | (79%) | (90%) | (36%) |
| Female | 23 | 29 | 25 | 14 | 3 | 78 |
| | (24%) | (27%) | (30%) | (21%) | (10%) | (65%) |

Age

| | | | | | |
|---|---|---|---|---|---|
| 55.1 | 55.0 | 57.0 | 54.8 | 63.4 | 56.2 |
| (13.3) | (11.7) | (10.8) | (10.0) | (13.9) | (11.5) |

HBV

| Yes | 71 | 85 | 63 | 50 | 24 | 29 |
|---|---|---|---|---|---|---|
| | (76%) | (78%) | (76%) | (74%) | (83%) | (24%) |
| No | 23 | 24 | 20 | 18 | 2 | 81 |
| | (24%) | (22%) | (24.1%) | (26%) | (7%) | (67%) |
| NA | 0 | 0 | 0 | 0 | 3 | 11 |
| | (0%) | (0%) | (0%) | (0%) | (10%) | (9%) |

AFP

| | | | | | | | |
|---|---|---|---|---|---|---|---|
| ≤20 | ng/ml | 29 | 97 | 43 | 66 | 10 | 107 |
| | | (31%) | (89%) | (52%) | (97%) | (35%) | (88%) |
| >20 | ng/ml | 65 | 12 | 40 | 2 | 15 | 3 |
| | | (69%) | (11%) | (48%) | (3%) | (52%) | (3%) |

| | | | | | | |
|---|---|---|---|---|---|---|
| NA | 0 | 0 | 0 | 0 | 4 | 11 |
| | (0%) | (0%) | (0%) | (0%) | (13%) | (9%) |

BCLC

| 0 + A | 28 | NA | 29 | NA | 6 | NA |
|---|---|---|---|---|---|---|
| | (30%) | | (35%) | | (21%) | |
| B + C + D | 65 | NA | 54 | NA | 21 | NA |
| | (70%) | | (65%) | | (73%) | |
| NA | 3 | NA | 0 | NA | 2 | NA |
| | (2%) | | (0%) | | (7%) | |

Tumor size

| | | | | | | | |
|---|---|---|---|---|---|---|---|
| ≤3 | cm | 34 | NA | 47 | NA | 9 | NA |
| | | (36%) | | (57%) | | (31%) | |
| >3 | cm | 57 | NA | 29 | NA | 17 | NA |
| | | (61%) | | (35%) | | (59%) | |

| | | | | | | |
|---|---|---|---|---|---|---|
| NA | 3 | NA | 7 | NA | 3 | NA |
| | (3%) | | (8.4%) | | (10%) | |

Macrovascular invasion

| Yes | 32 | NA | 30 | NA | 10 | NA |
|---|---|---|---|---|---|---|
| | (34%) | | (36%) | | (35%) | |
| No | 62 | NA | 53 | NA | 18 | NA |
| | (66%) | | (64%) | | (62%) | |
| NA | 1 | NA | 0 | NA | 1 | NA |
| | (1%) | | (0%) | | (3%) | |

Metastasis

| Yes | 14 | NA | 8 | NA | 2 | NA |
|---|---|---|---|---|---|---|
| | (15%) | | (9%) | | (7%) | |
| No | 97 | NA | 74 | NA | 26 | NA |
| | (84%) | | (90%) | | (90%) | |
| NA | 1 | NA | 1 | NA | 1 | NA |
| | (1%) | | (1%) | | (3%) | |

Data are n (%) or mean (SD).
Abbreviations: HCC, hepatocellular carcinoma; LC, liver cirrhosis; HBV, chronic hepatitis B virus; AFP, alpha-fetoprotein; BCLC, Barcelona Clinic Liver Cancer; SD, standard deviation; NA, not available.

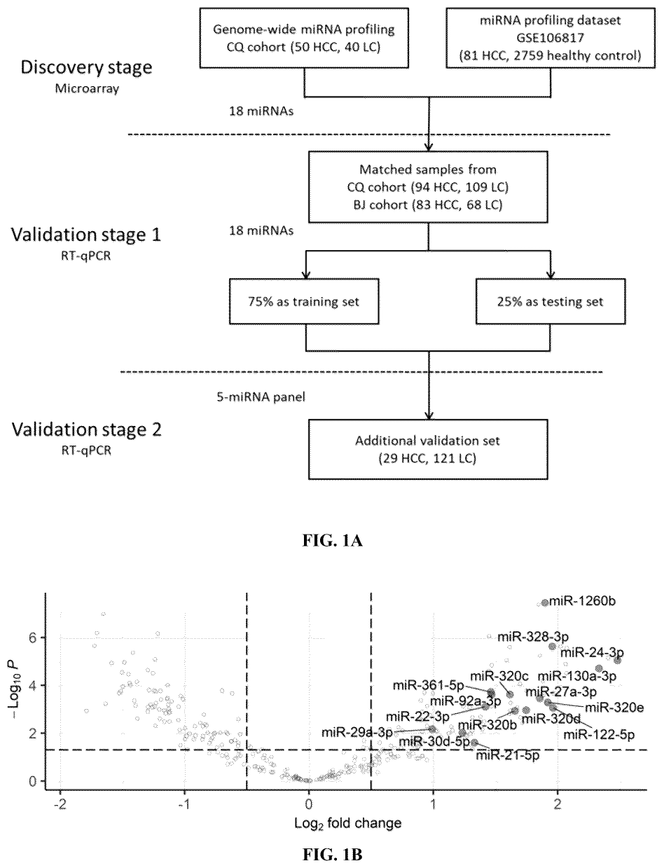

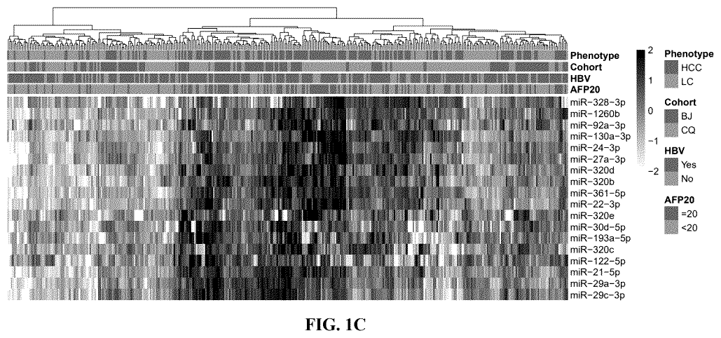

As illustrated in FIG. 1A, the workflow for identifying HCC-associated miRNAs consists of three key phases: a discovery stage, a first validation stage, and a second validation stage.

In the discovery stage, The Agilent Human miRNA Microarray Kit, Release 21.0, 8×60K (DesignID: 070156) experiment and data analysis are conducted by OE Biotechnology Co., Ltd. (Shanghai, China), according to the Agilent miRNA Microarray System with miRNA Complete Labeling and Hyb Kit protocol (Agilent Technologies). The slides are scanned with the Agilent scanner G2505C (Agilent Technologies). Raw data is extracted using Feature Extraction software (version 10.7.1.1, Agilent Technologies). Only miRNAs with detected signal in at least 50% of any sample group are included in further data analysis. The included data is normalized using the quantile normalization. As a result, 2,549 miRNAs are analyzed, among which 188 miRNAs demonstrate statistically significant differential expression between HCC and LC patients (P_{adj}<0.05) (FIG. 1B).

Cross-referencing these results with the publicly available miRNA profiling dataset GSE106817 leads to the identification of 16 consistently upregulated miRNAs (Table 2). Additionally, two previously reported miRNAs are included for further validation, resulting in a panel of 18 candidate targets including: miR-122-5p, miR-1260b, miR-130a-3p, miR-193a-5p, miR-21-5p, miR-22-3p, miR-24-3p, miR-27a-3p, miR-29a-3p, miR-29c-3p, miR-30d-5p, miR-320b, miR-320c, miR-320d, miR-320e, miR-328-3p, miR-361-5p, and miR-92a-3p (FIG. 1C).

In validation stage 1, the expression levels of these 18 candidate miRNAs are quantified using reverse transcription quantitative polymerase chain reaction (RT-qPCR) in 354 clinically matched HCC and LC samples. Briefly, 3 μl of extracted miRNA is reverse transcribed using the miRCURY LNA RT Kit (Qiagen) in 10 μl reactions. cDNA is diluted 20× before qPCR. qPCR is performed using miRCURY LNA miRNA Probe PCR Assays (Qiagen). The amplification conditions consist of a heat activation step at 95° C. for 2 mins, followed by 40 cycles of 5 s at 95° C. and 30 s at 56° C. qPCR reactions are run on the QuantStudio 7 Pro Real-Time PCR System (Applied Biosystems). A cycle threshold (Ct) value of 40 is imputed for the undetected reactions. miR-16-5p is used as an endogenous control for the normalization, and relative quantifications of miRNAs are calculated by 2^{-ΔCt}, where ΔCt=Ct_{target}−Ct_{control}. To ensure consistent quantifications throughout all reactions, three interplate controls are included in each PCR reaction to account for plate-to-plate variation. The results of each reaction are normalized against the interplate controls.

miRNA expressions from RT-qPCR are standardized by z-score normalization. Differential expression analyses are performed using either a two-sided Student's t-test or Wilcoxon rank sum test. A p-value of <0.05 and fold change >|2| are considered as statistically significant. P-values are adjusted for multiple hypothesis testing using the Benjamini-Hochberg method. Propensity score matching (PSM) is employed to select matched samples in validation stage 1 based on sex, age and HBV status as key confounding factors, resulting in 177 matched pairs of HCC and LC patients. To ensure that the above sample size has sufficient power to identify differential miRNAs expressions, a statistical power analysis is performed using the software G*Power. Considering the average effect sizes of significant miRNAs from validation stage 1, the effect size Cohen's D of miRNAs is set to be 0.69. The sample size of 177 pairs exceeds the minimum requirement of 47 paired samples for identifying differential miRNA expressions under a power of 80% and Type I error of 5%.

To ensure accurate relative quantification, the selection of an appropriate internal control is critical. Among the candidates tested, miR-16-5p demonstrates consistently high expression across all samples and shows a strong positive correlation with total miRNA levels (Pearson's r=0.82, P<0.001). Importantly, no significant expression differences are observed between the HCC and LC groups for miR-16-5p, validating its suitability as a normalization reference over traditional spike-in controls. After normalization with miR-16-5p, all 18 candidate miRNAs are confirmed to be significantly upregulated in HCC samples during validation stage 1, including the 16 originally identified in the discovery microarray analysis (FIG. 1B). These miRNAs (miR-122-5p, miR-320d, miR-1260b, miR-92a-3p, miR-21-5p, miR-130a-3p, miR-22-3p, miR-574-3p, miR-328-3p, miR-29c-3p, miR-27a-3p, miR-361-5p, miR-29a-3p, miR-320c, miR-24-3p, miR-423-5p, miR-148a-3p, miR-320b, miR-30d-5p, miR-193a-5p, and miR-320c) are detectable in over 92% of the samples and show no notable correlation with HBV status, with the exception of miR-122-5p. Notably, miR-361-5p, miR-130a-3p, and miR-24-3p emerge as the most significantly upregulated miRNAs, each with P_{adj} values below 10-15. Furthermore, unsupervised hierarchical clustering reveals that the samples cluster primarily according to miRNA expression profiles, independent of AFP levels or cohort origin (FIG. 1C). These results underscore the detection potential of the 18-miRNA panel as robust biomarkers capable of distinguishing HCC from LC patients based on molecular expression signatures.

### Example 2: Validating the miRNA Targets Through Meta-Analysis

Literature search in PubMed and Web of Science identifies a total of 221 publications relevant to circulating miRNAs for HCC diagnosis ( ) A systematic literature search is conducted in PubMed and Web of Science up to Dec. 14, 2023, to identify studies on HCC-related circulating miRNAs. The search utilized a combination of keywords and medical subject headings including “hepatocellular carcinoma”, “circulating microRNA”, “diagnosis” and “HBV”. Additional manual search is performed using references from retrieved articles and relevant reviews. Only articles published in English and peer-reviewed journals are considered. After removing duplicate records, the abstracts are screened to identify relevant articles. Studies are included based on the following criteria: (1) original studies; (2) clinical studies evaluating circulating miRNAs for the diagnosis of HCC; (3) miRNA profiling studies using serum or plasma as specimens. Following a thorough full-text review, studies are excluded if they do not meet the following criteria: (1) not a case-control study; (2) do not employ RT-qPCR as the quantification method; (3) include patients with viral co-infection or viral infection other than HBV as control.

Data extracted from eligible studies includes first author, year of publication, region, specimen type, sample size, sample characteristics, study design, normalization control, examined miRNAs, types of dysregulations, effect sizes and p-values. For studies that include comparisons against multiple control groups, effect size data versus the control group with higher risk of HCC is extracted (HBV-LC>LC>HBV). Both significant and non-significant results are included in the meta-analysis. If the effect size data is not directly reported, expression data is obtained from the graphical plot using PlotDigitizer. The following conservative conversions are applied to p values reported in a predefined significance threshold: p≥0.05 and p≥0.01 are converted to p=0.5, p<0.05 to p=0.025, p<0.01 to p=0.005, p<0.001 to p=0.0005, and p<0.0001 or p=0.0000 to p=0.00005. All relevant studies are assessed by two independent researchers. Any discrepancy is resolved through consultation with the lead researcher.

After full text assessment, 41 publications meet the inclusion criteria. A total of 93 differentially expressed miRNAs has been reported, most of which are identified in single studies only. Among them, 11 miRNAs with available expression data from 3 or more studies are eligible for meta-analysis, including miR-122-5p, miR-21-5p, miR-192-3p, miR-223-3p, miR-27a-3p, miR-26a-5p, miR-29a-3p, miR-29c-3p, miR-193a-5p, miR-125b-5p, and miR-214-3p. Among these 18 miRNA targets, 5 of them have been reported as upregulated in HCC.

Specifically, the effect sizes and corresponding measures of variance (p value, 95% confidence intervals [95% CIs], or standard errors) are extracted from included studies. If means and standard deviations are not provided, they are calculated from the corresponding measure of variance or estimated from the median and interquartile range. Whenever the effect sizes of a miRNA are available from three or more studies, Hedges' g is calculated as pooled standardized mean differences (SMD) between HCC and LC patients for the meta-analysis. Heterogeneity among the studies is assessed using Cochran's Q test and Higgin's inconsistency index (12). A P-value of less than 0.05 in the Cochran's Q test indicates significant heterogeneity. The DerSimonian-Laird random-effects model is applied for miRNAs with significant heterogeneity; otherwise, a fixed-effects model is used. Meta-analyses and heterogeneity tests are performed in R with the package “metafor” (version 4.2.0). The SMD and 95% CIs from the meta-analyses are presented with forest plots. Baujat plot and influence analysis are employed to identify studies with extreme effect sizes within the meta-analysis. Subgroup analyses are performed based on differences in experiment design. Normalization controls frequently used across multiple studies are classified as common controls, while RNU6 is classified as a distinct group and other controls are classified as less common controls. Potential publication bias is examined by visual inspection of the funnel plot and Egger's test. Unless otherwise specified, a two-sided P value of less than 0.05 is considered statistically significant. Significance in the meta-analysis is adjusted for multiple testing using Bonferroni correction (ie, α=0.05/11=4.55×10⁻³).

miR-122-5p and miR-21-5p emerge as the most frequently reported miRNAs among those identified, establishing them as leading candidates for meta-analysis. However, initial meta-analyses that include all available studies reveal significant heterogeneity for both miRNAs, resulting in inconclusive outcomes. No evidence of publication bias is detected for either miRNA. Baujat plot analysis identifies key sources of heterogeneity, which are largely attributed to studies employing less commonly used normalization controls. In particular, the study by Xu et al. contributes substantially to the heterogeneity for miR-122-5p, while the study by Guo et al. does so for miR-21-5p.

To address this issue, studies are stratified into subgroups based on the normalization controls utilized. This subgrouping markedly reduces heterogencity within each group. In studies that use either miR-16-5p or synthetic spike-in controls, consistent upregulation of miR-122-5p (effect size=0.66, 95% CI: 0.53-0.78; P=1.17×10⁻²⁴) and miR-21-5p (effect size=0.41, 95% CI: 0.04-0.78; P=0.028) is observed.

Applying the same subgroup-based approach to additional miRNAs, significant upregulation of miR-192-3p and miR-29a-3p is identified in studies utilizing common normalization controls. In contrast, meta-analyses of studies using mixed or less standardized controls reveal one consistently upregulated miRNA (miR-193a-5p) and two consistently downregulated miRNAs (miR-26a-3p and miR-223-3p).

Collectively, the meta-analysis results validated the upregulation of four miRNAs-miR-122-5p, miR-21-5p, miR-29a-3p, and miR-193a-5p-previously identified in the platform, thereby reinforcing the robustness and reproducibility of the miRNA profiling methodology for detecting dysregulated miRNAs in HCC.

### Example 3: Establishment and Validation of a Combined 5-miRNA and AFP20 Panel for HCC Detection

Univariate logistic regression analysis is first conducted to assess the detection performance of individual miRNAs in distinguishing HCC from liver cirrhosis LC. Briefly, matched samples in validation stage 1 are randomly split into a training set (75%) and testing set (25%). Following 100 iterations of repeated 5-fold cross-validation, LASSO regression analysis is performed to establish an optimal miRNA panel with AFP20 in the training set using the caret package. The miRNA set with the highest AUC is selected as the final panel for further validation in the testing set and additional validation set. Multicollinearity among variables in the panel is examined by variance inflation factor, with a score of <5 indicating acceptable multicollinearity. Risk scores are calculated for all patients, defined as the sum of each miRNA expression weighted by its corresponding logistic regression coefficient in the model. The risk score cutoff is determined by Youden's index to differentiate patients into high-risk and low-risk groups. Subgroup analyses are performed across multiple sample subsets to evaluate the robustness and the detection performance of the established miRNA panel. Detection performance, sensitivity and specificity of the models are evaluated using AUC and receiver operating characteristics (ROC) analysis. PSM is performed using the matchit function in the MatchIt package. Statistical comparisons of ROC curves are performed using Delong's test in the pROC package. Statistical analyses are performed using R, version 4.4.1.

Among the 18 upregulated miRNAs, miR-361-5p demonstrates the highest area under the curve (AUC) at 0.801, while AFP20 yields an AUC of 0.767. Several top-ranked miRNAs show detection performance comparable to AFP20, whereas the remainder generally exhibits lower accuracy. Importantly, none of the 18 miRNAs shows a significant correlation with AFP levels (Pearson correlation, all P>0.05), indicating that the expression of these miRNAs and AFP likely occurs through independent biological pathways. This independence supports the potential utility of circulating miRNAs as complementary biomarkers to AFP in the detection of HCC.

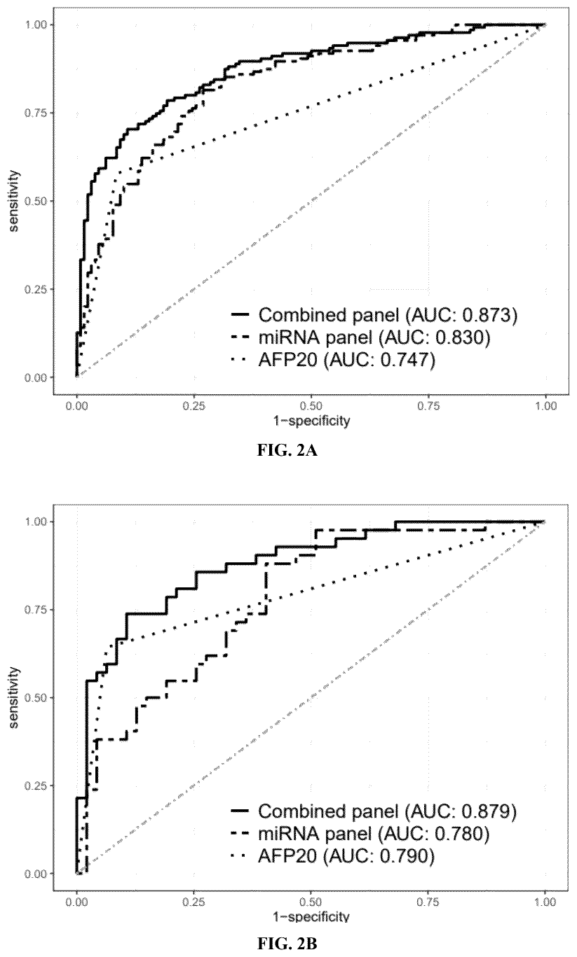

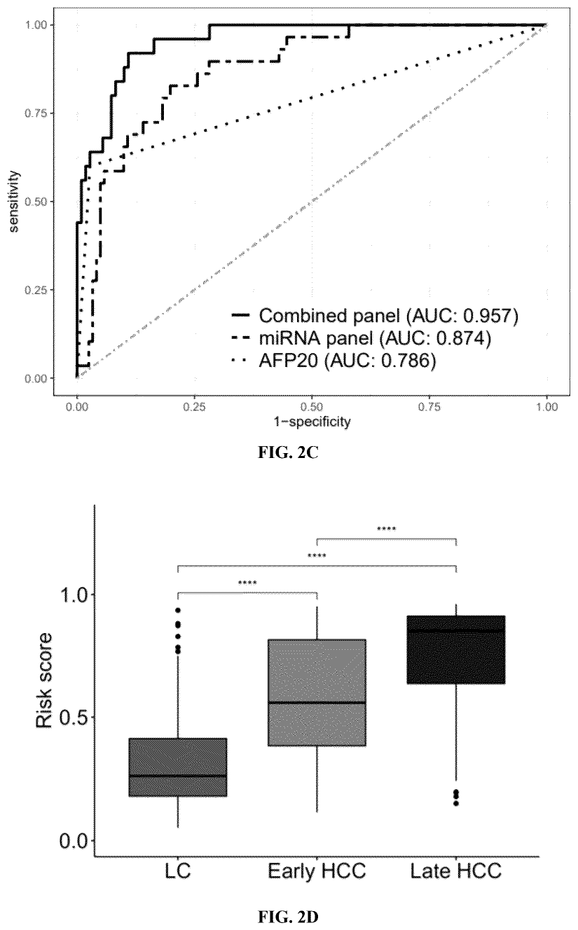

To further enhance detection accuracy, an explainable machine learning approach is employed to integrate multiple miRNA signals. A logistic least absolute shrinkage and selection operator (LASSO) regression model is trained using 75% of the 354 matched samples. The analysis identifies an optimal miRNA panel composed of five miRNAs—miR-361-5p, miR-130a-3p, miR-27a-3p, miR-30d-5p, and miR-193a-5p—in combination with AFP20. This integrated panel achieves an AUC of 0.873 (95% CI: 0.831-0.915) in the training set (FIG. 2A). When applied to the remaining 25% of samples (testing set), the panel maintains strong performance with an AUC of 0.879 (95% CI: 0.809-0.950) (FIG. 2B). Evaluation in an independent validation set further confirms its robustness, achieving an AUC of 0.957 (95% CI: 0.924-0.990) (FIG. 2C). In contrast, AFP20 alone demonstrates consistently lower performance across all datasets (AUC=0.747 [95% CI: 0.698-0.795] in the training set; 0.790 [95% CI: 0.708-0.870] in the testing set; and 0.786 [95% CI: 0.687-0.886] in the validation set; FIGS. 2A-2C).

Further subgroup analyses are performed to assess detection performance across clinically relevant HCC subsets, including early-stage HCC, HBV-associated HCC, HBV-negative HCC, and AFP-negative HCC. The integrated panel consistently outperformed AFP20 alone in all subsets. Notably, the combined panel also demonstrates superior detection performance over continuous AFP values in the training set. A summary of detection metrics for overall and early-stage HCC is provided in Table 2.

TABLE 2
Comparison of the detection performance of combined
panel and AFP20 to identify patients with HCC or
early-stage HCC in the testing and validation set

| | | |
|---|---|---|
| Combined panel | AFP20 | p-val |

Detecting overall HCC (67 HCC vs 157 LC)

| | | | |
|---|---|---|---|
| Cutoff | 0.557 | 0.500 | |
| AUC (95% CI) | 0.924 (0.887-0.960) | 0.794 (0.734-0.855) | 6.2 × 10⁻⁸ |
| Sensitivity | 0.776 | 0.627 | |
| Specificity | 0.911 | 0.962 | |

Detecting early-stage HCC (24 early-stage HCC vs 157 LC)

| | | | |
|---|---|---|---|
| AUC (95% CI) | 0.876 (0.803-0.949) | 0.731 (0.628-0.834) | 5.57 × 10⁻⁴ |
| Sensitivity | 0.625 | 0.500 | |
| Specificity | 0.911 | 0.962 | |

Early-stage HCC is defined as patients with BCLC stage 0 and A. The comparison is performed in the same set of patients. Samples with missing AFP level are excluded. DeLong's test is used to compare the AUC of combined panel and AFP20.

To stratify patient risk, a composite risk score is calculated for each subject. A progressive increase in risk score is observed with advancing HCC stage (FIG. 2D), while no significant differences are noted when stratified by age, sex, or HBV status. Using Youden's index as the classification threshold (0.5572), both the combined panel and AFP20 achieve specificity greater than 90% for early-stage HCC detection. However, the integrated panel shows markedly improved sensitivity compared to AFP20 alone (63% vs. 50%), underscoring its potential to enhance screening efficacy and early detection of HCC when used in combination with AFP.

### Example 4: Associations of miRNAs with Clinical Parameters and Target Genes

The association between miRNA expression levels and key tumor-related clinical characteristics in HCC patients is further investigated. Target genes of miRNAs are predicted using 3 platforms, TargetScan8.0, miRDB and miRWalk. Functional enrichment of mutually predicted target genes is performed using the Kyoto Gene and Genome Encyclopedia (KEGG) database. Predicted targets from 3 or more miRNAs are used to construct the interaction networks using Cytoscape v3.10.2.

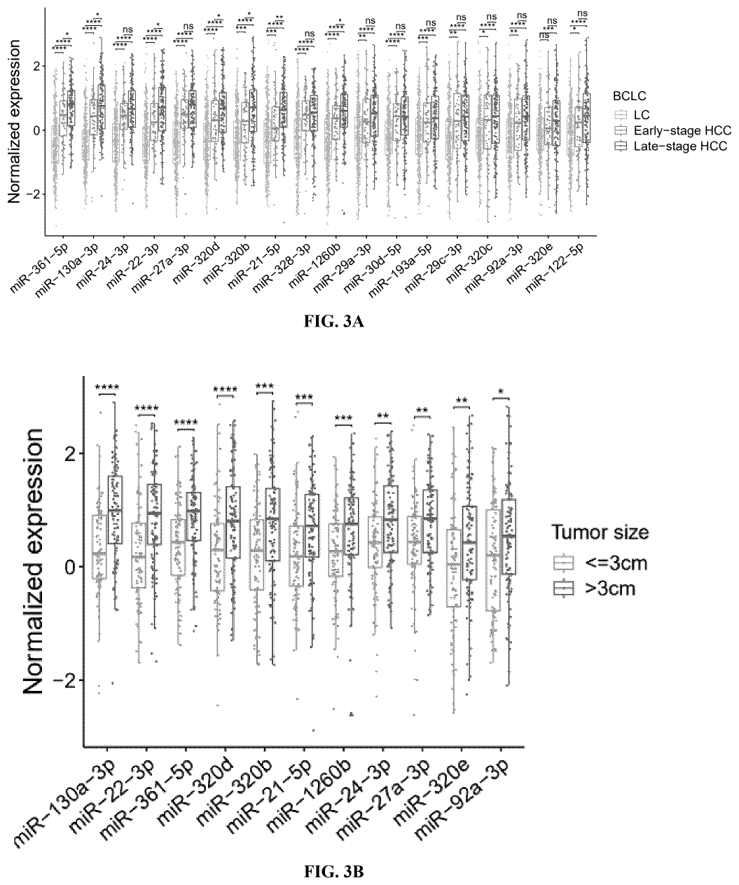

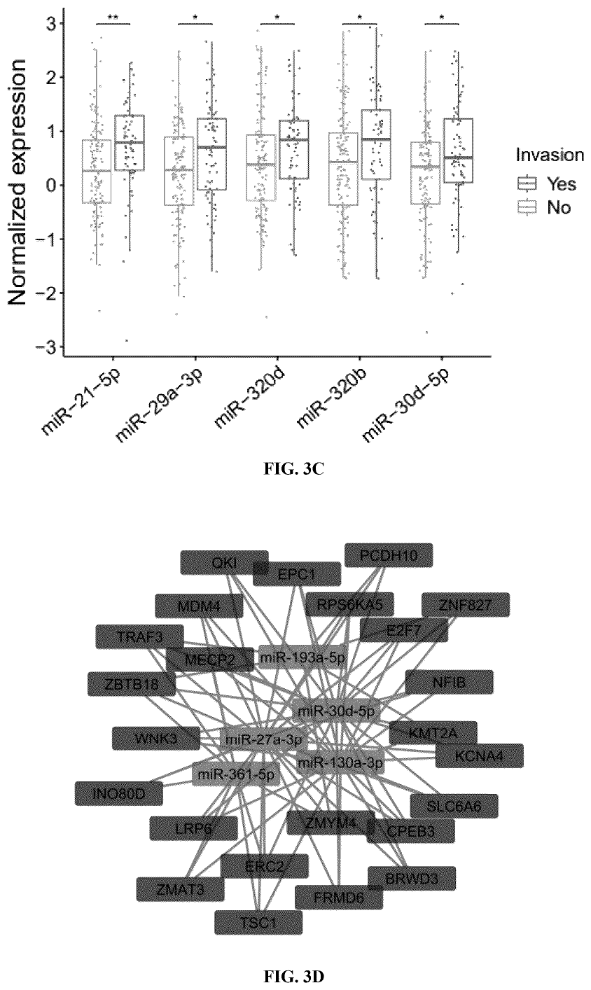

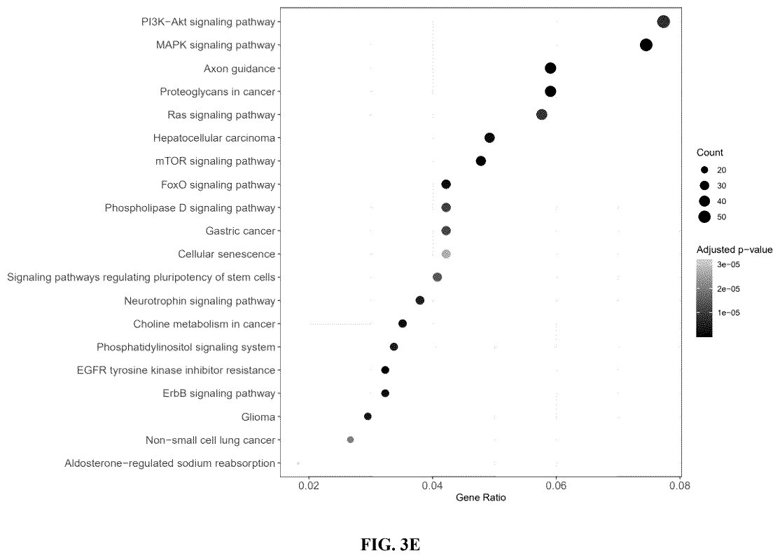

Consistent with the strong performance of the identified miRNA panel for early-stage HCC, 17 miRNAs are found to be significantly upregulated in patients with early-stage disease (FIG. 3A), reinforcing their potential utility as biomarkers for early detection. Moreover, 11 miRNAs exhibit increased expression in patients with larger tumor sizes (FIG. 3B), and 5 miRNAs are upregulated in cases with evidence of tumor invasion (FIG. 3C).

Among the five miRNAs included in the present miRNA panel, three-miR-130a-3p, miR-361-5p, and miR-27a-3p—demonstrate expression patterns positively correlated with advancing tumor stage and tumor size, suggesting their potential relevance not only for detection but also for disease stratification.

To explore the potential molecular mechanisms underlying the dysregulation of these miRNAs, three bioinformatics tools—TargetScan 8.0, miRDB, and miRWalk—are used to predict their target genes. The overlapping gene targets identified across all three platforms are integrated to construct an interaction network (FIG. 3D). Subsequent KEGG pathway enrichment analysis of these overlapping targets reveals significant enrichment in key oncogenic signaling pathways, including the PI3K-Akt, MAPK, and HCC pathways (FIG. 3E), suggesting that the dysregulated miRNAs may contribute functionally to tumor progression through these signaling cascades.

The present invention achieves notable advancements in detecting a HCC-related panel of circulating miRNAs with AFP20 through liquid biopsy within at-risk populations. This strategy offers a significant improvement over current standard screening methods, which rely predominantly on AFP alone. Due to its limited sensitivity, especially for early-stage HCC, AFP-based screening frequently results in delayed detection and poor clinical outcomes.

In the present invention, 18 significantly upregulated circulating miRNAs are identified in HCC patients, four of which are further validated through comprehensive meta-analysis. From this group, a panel including five miRNAs—miR-361-5p, miR-130a-3p, miR-27a-3p, miR-30d-5p, and miR-193a-5p—combined with/without AFP20 is developed. This panel demonstrates excellent performance in distinguishing HCC from LC, including in patients with early-stage disease. Importantly, the five-miRNA panel also retains standalone detection utility for HCC screening in patients without available AFP data. The present invention represents one of the largest high-throughput, multicenter investigations of circulating miRNAs detection, highlighting the promise of miRNA-based liquid biopsy as a minimally invasive tool for early cancer detection. While it is not intended to replace imaging-based detection approaches, this method offers a valuable adjunct that could enhance HCC surveillance by serving as a first-line screening modality in combination with AFP.

Among the miRNAs analyzed, miR-122-5p and miR-21-5p emerge as the most frequently reported in previous literature and meta-analyses, reinforcing their detection potential. miR-122-5p, a liver-specific miRNA, has been extensively studied, though its circulating levels in HCC patients have shown contradictory results. The meta-analysis suggests that this discrepancy is largely attributable to differences in normalization strategies across studies. Moreover, circulating miR-122-5p levels are not unique to HCC and have also been associated with other chronic liver conditions, including HBV infection, hepatitis C, NAFLD, and drug-induced liver injury. In contrast, hepatic miR-122-5p expression is consistently downregulated in HCC tissues.

Interestingly, the present invention reveals an inverse relationship between circulating and hepatic miR-122-5p levels. Upregulation of circulating miR-122-5p shows little or no correlation with traditional liver injury markers such as aspartate aminotransferase (AST) or alanine aminotransferase (ALT) (r=0.10, P=0.03 for AST; r=0.03, P>0.05 for ALT), suggesting that its increase in blood is not solely attributable to hepatocyte death. A similar phenomenon has been observed in non-alcoholic steatohepatitis (NASH), where Pirola et al. proposed that reduced hepatic miR-122-5p levels could result from active export into the circulation.

Other key miRNAs in the miRNA panel of the present invention, including miR-361-5p, miR-130a-3p, and miR-27a-3p, have not been widely reported in prior HCC studies. Of note, miR-27a-3p has been shown to mediate intergenerational HCC susceptibility in animal models; for example, obesity-induced upregulation of serum miR-27a-3p during pregnancy increased hepatic expression and HCC risk in the offspring, underscoring its possible role in tumorigenesis.

For normalization, miR-16-5p is selected due to its stable and high expression across the study cohort. This is further supported by meta-analysis showing consistent quantification across studies using miR-16-5p as a control. However, it is worth noting that some studies have reported downregulation of miR-16-5p in HCC, suggesting the need for further evaluation and consensus regarding optimal normalization strategies for circulating miRNA assays.

In summary, the present invention systematically identifies and validates a five-miRNA panel combined with AFP20. The combined panel demonstrates superior performance across diverse clinical subgroups, including early-stage HCC, HBV-associated HCC, and AFP-negative HCC. These results underscore the potential utility of the panel as a reliable, non-invasive, and scalable tool for population-based HCC screening and early cancer detection.

The functional units and modules of the systems and methods in accordance with the embodiments disclosed herein may be implemented using computing devices, computer processors, or electronic circuitries including but not limited to application specific integrated circuits (ASIC), field programmable gate arrays (FPGA), microcontrollers, central processing units (CPU), graphical processing units (GPU), and other programmable logic devices configured or programmed according to the teachings of the present disclosure. Computer instructions or software codes executing in the computing devices, computer processors, or programmable logic devices can readily be prepared by practitioners skilled in the software or electronic art based on the teachings of the present disclosure.

All or portions of the methods in accordance with the embodiments may be executed in one or more computing devices including server computers, personal computers, laptop computers, mobile computing devices such as smartphones and tablet computers.

The embodiments may include computer storage media, transient and non-transient memory devices having computer instructions or software codes stored therein, which can be used to program or configure the computing devices, computer processors, or electronic circuitries to perform any of the processes of the present invention. The storage media, transient and non-transient memory devices can be included, but are not limited to, floppy disks, optical discs, Blu-ray Disc, DVD, CD-ROMs, and magneto-optical disks, ROMs, RAMs, flash memory devices, or any type of media or devices suitable for storing instructions, codes, and/or data.

Each of the functional units and modules in accordance with various embodiments also may be implemented in distributed computing environments and/or Cloud computing environments, wherein the whole or portions of machine instructions are executed in distributed fashion by one or more processing devices interconnected by a communication network, such as an intranet, Wide Area Network (WAN), Local Area Network (LAN), the Internet, and other forms of data transmission medium.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

1. A system for diagnosing hepatocellular carcinoma (HCC) in a subject, comprising:
a sample preparation module configured to receive a blood sample and isolate a plasma fraction and a serum fraction therefrom;
a nucleic acid extraction module configured to extract a miRNA profile from the plasma fraction;
a real-time PCR module configured to detect and quantify expression levels of a panel of microRNAs comprising miR-361-5p, miR-130a-3p, miR-27a-3p, miR-30d-5p, and miR-193a-5p in the miRNA profile;
a computing device comprising a processor and memory storing instructions that, when executed, cause the processor to:
receive the quantified expression levels of the panel of microRNAs;
process the quantified expression levels of the panel of microRNAs using a trained classifier to generate a HCC risk classification categorizing the subject into one of HCC risk levels.

2. The system of claim 1, wherein the HCC risk levels comprise low HCC risk, HCC indeterminate risk, and high HCC risk.

3. The system of claim 1, wherein the computing device is further configured to:
receive an alpha-fetoprotein (AFP) level detected and quantified from the serum fraction; and
compare the received AFP level to a threshold of 20 ng/mL.

4. The system of claim 3, wherein the trained classifier is configured to incorporate the received AFP level as a binary input, together with the expression levels of the panel of miRNAs, in generating the HCC risk classification regardless of whether the AFP level is below or above the threshold, and wherein, when the AFP level is unavailable, the classifier is configured to classify the HCC risk based solely on the expression levels of the panel of miRNAs.

5. The system of claim 1, wherein the trained classifier comprises a logistic regression model trained using annotated training datasets comprising microRNA expression profiles and ground-truth HCC diagnoses.

6. The system of claim 1, wherein the system further comprises a training module configured to update the trained classifier based on new annotated training datasets.

7. The system of claim 1, wherein the subject is a liver cirrhosis patient.

8. A computer-implemented method for generating a HCC report of a subject, comprising:
obtaining a blood sample from a subject;
isolating a plasma fraction from the blood sample;
extracting a miRNA profile of the subject from the plasma fraction;
detecting and quantifying the expression levels of a panel of microRNAs comprising miR-361-5p, miR-130a-3p, miR-27a-3p, miR-30d-5p, and miR-193a-5p from the miRNA profile;
processing the quantified expression levels with a trained machine learning classifier implemented on a computer processor, wherein the machine learning classifier is configured to generate a HCC risk classification categorizing the subject into one of HCC risk levels.

9. The method of claim 8, further comprising:
detecting and quantifying an AFP level from a serum fraction isolated from the blood sample; and
comparing the AFP level to a threshold of 20 ng/mL.

10. The method of claim 9, wherein the trained machine learning classifier is further configured to incorporate the received AFP level as a binary input, together with the expression levels of the panel of miRNAs, in generating the HCC risk classification regardless of whether the AFP level is below or above the threshold, and wherein, when the AFP level is unavailable, the classifier is configured to classify the HCC risk based solely on the expression levels of the panel of miRNAs.

11. The method of claim 8, wherein the subject is a liver cirrhosis patient.

12. The method of claim 8, wherein the classifier comprises a logistic regression model trained using LASSO regularization on annotated training datasets.

13. The method of claim 8, wherein the HCC report further comprises one or more indications of: repeat testing in 3 months, ordering a multiphase contrast-enhanced MRI, or referring for oncology consultation.
